(19) United States
(12) Patent Application Publication
(10) Pub. No.: US 2026/0203901 A1
(43) Pub. Date: Jul. 16, 2026

(54) DEVICE FOR GENERATING VIRTUAL CONTOUR OF BLOOD VESSEL AND METHOD OF GENERATING THE SAME

(71) Applicant: Medipixel, Inc., Seoul (KR)

(72) Inventors: Junsup SHIN, Seoul (KR); Young-Eon KIM, Seoul (KR)

(21) Appl. No.: 19/445,094

(22) Filed: Jan. 9, 2026

(30) Foreign Application Priority Data
Jan. 13, 2025 (KR) 10-2025-0004668
Mar. 25, 2025 (KR) 10-2025-0038326

(51) Int. Cl. G06T 7/00; A61B 6/50; G06T 7/13; G06T 7/564; G06T 7/62; G06T 7/68

(52) U.S. Cl. CPC G06T 7/0012; G06T 7/13; G06T 7/564; G06T 7/62; G06T 7/68; A61B 6/504; G06T 2207/20072; G06T 2207/30104

(57) ABSTRACT

An electronic device for providing vascular information from a medical image is disclosed. The device extracts a contour of a target blood vessel including a first branch and a plurality of second branches. A first reference point is determined where a contour of a target branch connects to a contour of another branch. A first candidate contour is generated by extending the target branch contour from the first reference point to a second reference point at a boundary between the other branch and the first branch. One or more second candidate contours are generated by extending the target branch contour from the first reference point to points different from the second reference point. A target contour for a path along the first branch and the target branch is determined based on the candidate contours. Vascular information, including a diameter and a shape, is provided based on the target contour.

### CROSS-REFERENCE TO RELATED APPLICATIONS

This application claims the benefit of Korean Patent Application No. 10-2025-0004668, filed on Jan. 13, 2025, and Korean Patent Application No. 10-2025-0038326, filed on Mar. 25, 2025, in the Korean Intellectual Property Office, the entire disclosures of which are incorporated herein by reference for all purposes.

### BACKGROUND

### 1. Field of the Invention

One or more embodiments relate to a technology for generating a virtual contour in a branch region of a blood vessel.

### 2. Description of the Related Art

A technology for extracting and analyzing a blood vessel from a medical image may be used for diagnosis and treatment planning of coronary artery disease, peripheral vascular disease, and neurovascular disease. For example, a structure of a blood vessel may be quantitatively analyzed using a medical imaging technology such as angiography. However, in a region where a blood vessel branches, a vascular diameter changes non-linearly depending on each branch direction, and accurate diameter measurement may be difficult. In particular, different hemodynamic conditions act in a vascular branch region, and thus it may be difficult to accurately reflect the shape of a blood vessel formed in a branch direction using existing diameter measurement techniques. Accordingly, a technology capable of more accurately measuring and analyzing a vascular diameter corresponding to each branch direction in a vascular branch region is required.

The above description has been possessed or acquired by the inventor(s) in the course of conceiving the present disclosure and is not necessarily an art publicly known before the present application is filed.

### SUMMARY

According to an aspect, there is provided an electronic device for medical image processing. The electronic device may include an image acquisition unit configured to acquire a medical image, memory configured to store the medical image and a plurality of instructions, and a processor configured to execute the plurality of instructions stored in the memory, wherein, when executing the plurality of instructions, the processor may cause the electronic device to extract a contour of a target blood vessel including a first branch and a plurality of second branches branching from the first branch from the medical image, determine a first reference point at which, among the plurality of second branches, a contour of a target branch is connected to a contour of another branch, generate a first candidate contour by extending the contour of the target branch from the first reference point to a second reference point that is a boundary between the other branch and the first branch, generate one or more second candidate contours by extending the contour of the target branch from the first reference point to another point that is different from the second reference point, determine a target contour corresponding to a path along the first branch and the target branch based on the first candidate contour and the one or more second candidate contours, and provide vascular information including a vascular diameter and a vascular shape of the path based on the target contour.

When executing the plurality of instructions, the processor may cause the electronic device to generate the one or more second candidate contours by extending the contour of the target branch from the first reference point to the other point located farther from the target branch than the second reference point.

When executing the plurality of instructions, the processor may cause the electronic device to generate the first candidate contour by linearly extending the contour of the target branch from the first reference point to the second reference point.

When executing the plurality of instructions, the processor may cause the electronic device to generate, based on a predetermined first partial contour of the target branch and a predetermined second partial contour of the first branch, the one or more second candidate contours by extending the predetermined first partial contour from the first reference point to the other point.

When executing the plurality of instructions, the processor may cause the electronic device to generate a vascular centerline of the target blood vessel based on the contour of the target blood vessel, generate one or more intermediate contours by extending the vascular centerline of the target branch in a direction from the first reference point toward the other point, and generate the one or more second candidate contours by transforming the one or more intermediate contours in a direction toward the contour of the target blood vessel.

When executing the plurality of instructions, the processor may cause the electronic device to, in response to moving the other point by a predetermined interval in a direction of the first branch, generate the one or more second candidate contours by extending the contour of the target branch from the first reference point toward the moved other point.

When executing the plurality of instructions, the processor may cause the electronic device to determine, among the one or more second candidate contours, the target contour that is located inside the first candidate contour.

When executing the plurality of instructions, the processor may cause the electronic device to extract diameter information of the target blood vessel corresponding to the one or more second candidate contours by scanning the target blood vessel in a direction from the first branch toward the target branch, and determine the target contour based on linearity of a graph including a position in the direction of the target branch relative to the first branch as a first axis and the diameter information of the target blood vessel as a second axis.

When executing the plurality of instructions, the processor may cause the electronic device to extract the contour corresponding to the target blood vessel by masking the target blood vessel in the medical image.

When executing the plurality of instructions, the processor may cause the electronic device to determine, as the first reference point, a point at which a distance from a predetermined branch point included in a branch region where the plurality of second branches branch from the first branch to the contour of the target branch is minimum, and determine, as the second reference point, an intersection point located at a minimum distance from the branch point among intersection points formed between the contour of the target blood vessel and vectors disposed between a vector in a direction from the branch point toward the other branch and a vector in a direction from the branch point toward the first branch.

When executing the plurality of instructions, the processor may cause the electronic device to generate a vascular centerline of the target blood vessel based on the contour of the target blood vessel, and determine a point at which the vascular centerline branches as a branch point.

When executing the plurality of instructions, the processor may cause the electronic device to calculate a Fractional Flow Reserve (FFR) corresponding to a blood vessel on the path based on the vascular information.

According to another aspect, there is provided a method of processing a medical image. The method may include extracting a contour of a target blood vessel including a first branch and a plurality of second branches branching from the first branch from a medical image, determining a first reference point at which, among the plurality of second branches, a contour of a target branch is connected to a contour of another branch, generating a first candidate contour by extending the contour of the target branch from the first reference point to a second reference point that is a boundary between the other branch and the first branch, generating one or more second candidate contours by extending the contour of the target branch from the first reference point to another point that is different from the second reference point, determining a target contour corresponding to a path along the first branch and the target branch based on the first candidate contour and the one or more second candidate contours, and providing vascular information including a vascular diameter and a vascular shape of the path based on the target contour.

The generating of the one or more second candidate contours may include generating the one or more second candidate contours by extending the contour of the target branch from the first reference point to the other point located farther from the target branch than the second reference point.

The generating of the first candidate contour may include generating the first candidate contour by linearly extending the contour of the target branch from the first reference point to the second reference point.

The generating of the one or more second candidate contours may include generating, based on a predetermined first partial contour of the target branch and a predetermined second partial contour of the first branch, the one or more second candidate contours by extending the predetermined first partial contour from the first reference point to the other point.

The generating of the one or more second candidate contours may include generating a vascular centerline of the target blood vessel based on the contour of the target blood vessel, generating one or more intermediate contours by extending the vascular centerline of the first branch in a direction from the first reference point toward the other point, and generating the one or more second candidate contours by transforming the one or more intermediate contours in a direction toward the contour of the target blood vessel.

The generating of the one or more second candidate contours may include, in response to moving the other point by a predetermined interval in a direction of the first branch, generating the one or more second candidate contours by extending the contour of the target branch from the first reference point toward the moved other point.

The determining of the target contour may include determining, among the one or more second candidate contours, the target contour that is located inside the first candidate contour.

Additional aspects of embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a schematic block diagram of an electronic device for medical image processing according to an embodiment.

FIG. 2 illustrates a first candidate contour generated by an electronic device according to an embodiment.

FIG. 3 illustrates a second candidate contour generated by an electronic device based on a contour of a target blood vessel according to an embodiment.

FIG. 4 illustrates a second candidate contour generated by an electronic device in a lesion region according to an embodiment.

FIG. 5 illustrates a second candidate contour generated by an electronic device based on a vascular centerline according to an embodiment.

FIG. 6 illustrates a plurality of candidate contours generated by an electronic device according to an embodiment.

FIG. 7 is a diagram for describing a method by which an electronic device determines a target contour according to an embodiment.

FIG. 8 is a diagram for describing a method by which an electronic device determines a target contour according to an embodiment.

FIG. 9 is a diagram for describing a method by which an electronic device determines a target contour according to an embodiment.

FIG. 10 is a diagram for describing a method by which an electronic device determines a target contour according to an embodiment.

FIG. 11 is a flowchart of a method of processing a medical image, performed by an electronic device, according to an embodiment.

### DETAILED DESCRIPTION

The following detailed structural or functional description is provided as an example only and various alterations and modifications may be made to the examples. Accordingly, the embodiments are not construed as limited to the disclosure and should be understood to include all changes, equivalents, and replacements within the idea and the technical scope of the disclosure.

Although terms, such as first, second, and the like are used to describe various components, the components are not limited to the terms. These terms should be used only to distinguish one component from another component. For example, a first component may be referred to as a second component, and similarly the second component may also be referred to as the first component.

It should be noted that if one component is described as being “connected”, “coupled”, or “joined” to another component, a third component may be “connected”, “coupled”, and “joined” between the first and second components, although the first component may be directly connected, coupled, or joined to the second component.

As used herein, the singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, the embodiments will be described in detail with reference to the accompanying drawings. When describing the embodiments with reference to the accompanying drawings, like reference numerals refer to like components and a repeated description related thereto will be omitted.

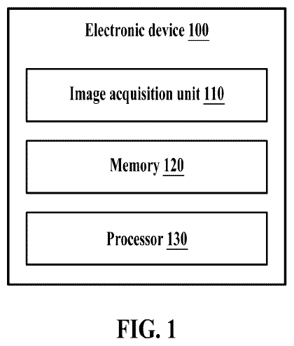

FIG. 1 is a schematic block diagram of an electronic device for medical image processing according to an embodiment.

An electronic device for medical image processing according to an embodiment (hereinafter, an electronic device 100) may analyze a blood vessel in a medical image. For example, the electronic device 100 may quantitatively and qualitatively analyze a blood vessel in a medical image. For example, the electronic device 100 may analyze a blood vessel structure included in a two-dimensional (2D) or three-dimensional (3D) medical image acquired from a medical imaging device such as X-ray, computed tomography (CT), or magnetic resonance imaging (MRI). For example, the electronic device 100 may identify blood vessels in the medical image. The electronic device 100 may analyze not only quantitative characteristics such as the diameter, length, and curvature of the blood vessels, but also qualitative characteristics such as branching structures of the blood vessels, presence or absence of stenosis, and morphological abnormalities. For example, the electronic device 100 may extract a vascular diameter for each path corresponding to each branch in a multi-branch region of a target blood vessel in a medical image. The electronic device 100 may accurately determine a vascular diameter of a scanned path by scanning the target blood vessel in a direction from a proximal portion toward a distal portion. In particular, since a plurality of paths is formed for each branch in a branch region, the electronic device 100 may obtain an estimated vascular diameter for each path in the branch region.

The electronic device 100 according to an embodiment may include an image acquisition unit 110, memory 120, and a processor 130.

For example, the electronic device 100 may acquire a medical image based on the image acquisition unit 110. The image acquisition unit 110 may acquire a medical image by directly capturing the medical image or may receive a medical image from an external device. For example, the image acquisition unit 110 may include hardware and/or software modules for acquiring a medical image including a blood vessel structure in a human body. For example, the image acquisition unit 110 may include a medical imaging device such as a CT device, an MRI device, an angiography device, or an ultrasound device, but is not limited thereto. In another example, the image acquisition unit 110 may be connected to an external medical imaging device in a wired or wireless manner and receive a medical image from the external medical imaging device. For example, the image acquisition unit 110 may receive an X-ray image in Digital Imaging and Communications in Medicine (DICOM) format captured from an external C-arm device. However, this is only an example of the image acquisition unit 110, and the image acquisition unit 110 is not limited to the examples listed above.

The memory 120 according to an embodiment may store a medical image and a plurality of instructions. For example, the memory 120 may store a medical image acquired through the image acquisition unit 110. For example, the memory 120 may include a non-volatile storage device. For example, the memory 120 may include a hard disk drive (HDD), a solid state drive (SSD), or a flash memory. In another example, the memory 120 may include a volatile memory device for temporary data processing. For example, the memory 120 may include dynamic random access memory (DRAM) or static random access memory (SRAM). However, this is only an example of the memory 120, and the memory 120 is not limited thereto. In addition, the memory 120 may store metadata (e.g., imaging date and time, patient information, imaging angle, etc.) corresponding to the medical image together. The memory 120 may store a plurality of instructions. For example, the memory 120 may include program code, algorithms, and control instructions executable by the processor 130.

The processor 130 according to an embodiment may execute a plurality of instructions stored in the memory 120. The processor 130 may determine a virtual contour for each branch in a branch region of a target blood vessel by executing a plurality of instructions stored in the memory 120.

For example, the processor 130 may extract a contour of a target blood vessel including a first branch and a plurality of second branches branching from the first branch from a medical image. For example, the medical image may include an X-ray acquired by administering a contrast agent to the target blood vessel. The target blood vessel may include the first branch and the plurality of second branches branching from the first branch. For reference, when the structure of the target blood vessel is represented as tree-type data, the first branch may represent a parent node and the plurality of second branches may represent child nodes of a node corresponding to the first branch. The first branch may be located at a proximal portion relative to a heart to which the target blood vessel is connected, and the second branches may be located at a distal portion relative to the first branch. The processor 130 may mask a target blood vessel in a medical image. For example, the processor 130 may mask a target blood vessel by adjusting a pixel value corresponding to the target blood vessel and pixel values corresponding to a background other than the target blood vessel. The processor 130 may extract a contour corresponding to the target blood vessel by masking the target blood vessel. For example, the contour corresponding to the target blood vessel may represent an outline of the target blood vessel.

The processor 130 according to an embodiment may determine a first reference point at which, among the plurality of second branches, a contour of a target branch is connected to a contour of another branch. For example, the processor 130 may select a target branch among the plurality of second branches. In other words, the processor 130 may select one target branch among the plurality of second branches to determine a vascular diameter of a path from the first branch to the corresponding second branch. The processor 130 may identify a point at which the contour of the target branch intersects with the contour of the other branch. For example, when the target branch and the other branch from the first branch in a Y-shape, the first reference point may represent an intersection point between the contour of the target branch and the contour of the other branch. Accordingly, the processor 130 may determine the intersection point between the contour of the target branch and the contour of the other branch as the first reference point.

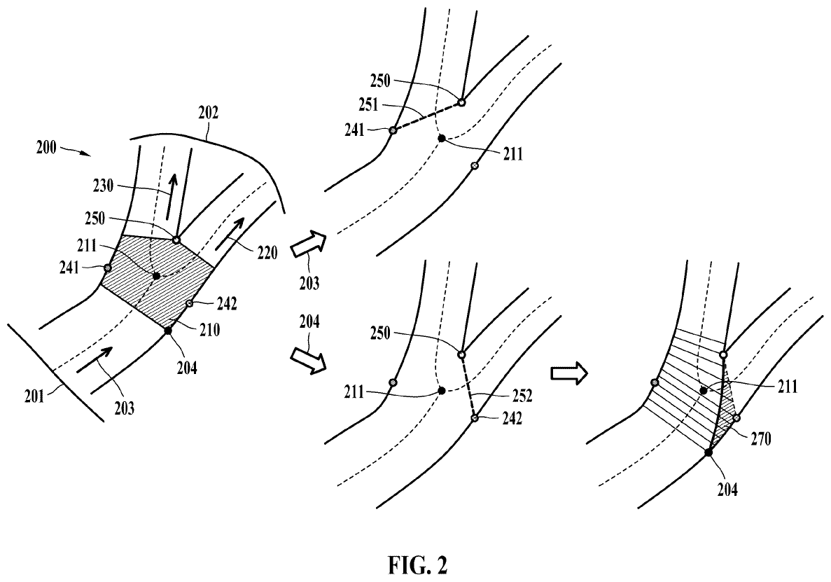

The processor 130 according to an embodiment may generate a first candidate contour by extending the contour of the target branch from the first reference point to a second reference point that is a boundary between the other branch and the first branch. For example, the processor 130 may determine the second reference point being the boundary between the other branch and the first branch. A method by which the processor 130 determines the second reference point will be described in detail below with reference to FIG. 2. For example, the processor 130 may extend, from the first reference point, a contour on the other branch side at the first reference point toward the determined second reference point. For example, the processor 130 may connect the first reference point to the second reference point with a straight line. A specific method by which the processor 130 generates the first candidate contour will be described in detail below with reference to FIG. 2.

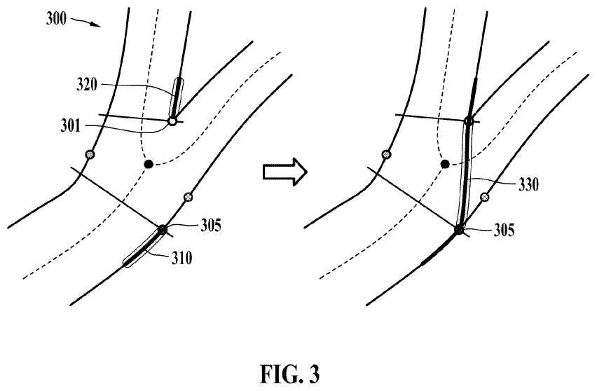

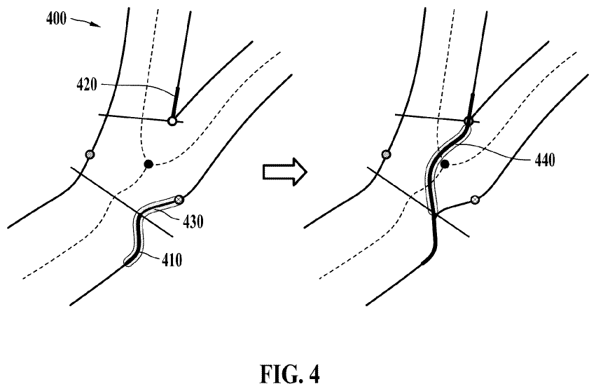

The processor 130 according to an embodiment may generate one or more second candidate contours by extending the contour of the target branch from the first reference point to another point that is different from the second reference point. For example, the other point that is different from the second reference point may represent a boundary point of a branch region of the target blood vessel predetermined by the processor 130. The processor 130 may distinguish between the branch region and other regions by generating a straight line perpendicular to the contour of the target blood vessel at a predetermined point on the first branch side. Similarly, the processor 130 may distinguish between the branch region and other regions by generating straight lines perpendicular to the contour of the target blood vessel at predetermined points on the plurality of second branches side. Here, a region surrounded by the straight line perpendicular to the contour at the predetermined point on the first branch side and straight lines perpendicular to the contour at the predetermined points on the second branches side may be referred to as the branch region. Accordingly, the processor 130 may determine a boundary on the first branch side among boundaries of the branch region as the other point that is different from the second reference point. The processor 130 may generate the one or more second candidate contours by extending the contour of the target branch to the other point. For example, the processor 130 may generate the one or more second candidate contours based on a Bezier curve. A detailed description of the second candidate contours generated by the processor 130 will be provided below with reference to FIGS. 3 to 4.

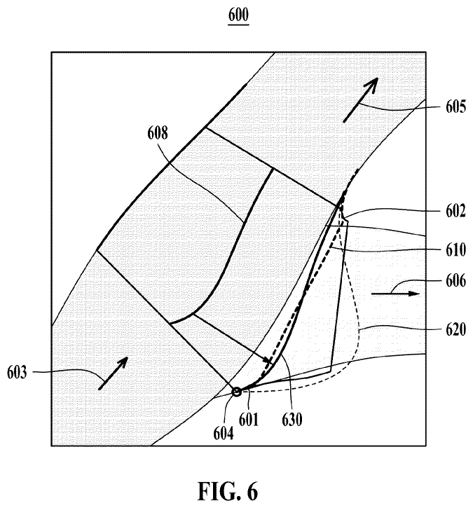

The processor 130 according to an embodiment may determine a target contour corresponding to a path along the first branch and the target branch based on the first candidate contour and the one or more second candidate contours. For example, the processor 130 may determine a contour that most naturally connects the first branch to the target branch as the target contour. For example, the processor 130 may compare the first candidate contour to the one or more second candidate contours. For example, the processor 130 may determine whether the first candidate contour and the one or more second candidate contours are located inside the target blood vessel or outside the target blood vessel. In addition, the processor 130 may determine whether the one or more second candidate contours are located inside the first candidate contour. The processor 130 may exclude a second candidate contour located outside the first candidate contour. The processor 130 may determine the first candidate contour as the target contour when all the second candidate contours are located outside the first candidate contour. In another example, when at least one second candidate contour is located inside the first candidate contour, the processor 130 may determine the target contour based on linearity of a vascular diameter corresponding to the second candidate contour. A method by which the processor 130 determines the target contour will be described in detail below with reference to FIGS. 6 to 10.

The processor 130 according to an embodiment may provide vascular information including a vascular diameter and a vascular shape of a path along the first branch and the target branch based on the target contour. For example, the processor 130 may generate the vascular information by analyzing data such as a change in the vascular diameter, a vascular curvature, and a blood flow in a branch region of the target blood vessel. For example, the processor 130 may calculate a Fractional Flow Reserve (FFR) corresponding to a blood vessel on a path along the first branch and the target branch based on the vascular information. The processor 130 may generate, based on the vascular information, a vascular diameter graph that changes linearly when scanning the target blood vessel from the first branch to the target branch. Accordingly, the processor 130 may obtain a highly reliable vascular diameter value even in the branch region.

Furthermore, the processor 130 may transmit the vascular information to a surgical robot device. For example, the processor 130 may control an operation in the branch region of the target blood vessel in conjunction with the surgical robot device through wired/wireless communication. For example, the processor 130 may assist the surgical robot device in determining the number of stents to be inserted into the branch region, sizes of the stents, and movement directions of the stents by providing the vascular information having accurately determined vascular diameters in the branch region to the surgical robot device.

FIG. 2 illustrates a first candidate contour generated by an electronic device according to an embodiment.

An electronic device according to an embodiment (e.g., the electronic device 100 of FIG. 1) may extract a contour 200 of a target blood vessel from a medical image. Referring to the contour 200 of the target blood vessel illustrated in FIG. 2, it is assumed that blood flows in a direction from a proximal portion 201 toward a distal portion 202 of the target blood vessel. The electronic device may divide the contour 200 of the target blood vessel into a plurality of regions by scanning the target blood vessel in a direction from the proximal portion 201 toward the distal portion 202 of the target blood vessel. For example, the electronic device may divide the contour 200 into a branch region 210 and other regions. For example, the contour 200 of the target blood vessel may include a first branch 203. In addition, the contour 200 of the target blood vessel may include a plurality of second branches 220 and 230 branching from the first branch 203. Here, the plurality of second branches 220 and 230 may branch from the first branch 203 in the branch region 210. The electronic device may distinguish between the branch region 210 and other regions by drawing a straight line perpendicular to the contour 200 at another point 204. The electronic device may determine a plurality of reference points 241, 242, and 250 included in the branch region 210. First, the electronic device may determine a first reference point 250 at which a contour of a target branch 230 is connected to a contour of another branch 220. In addition, the electronic device may determine a second reference point 242 that is a boundary between the other branch 220 and the first branch 203. Similarly, the electronic device may determine a third reference point 241 that is a boundary between the target branch 230 and the first branch 203. For example, the electronic device may determine the first to third reference points 241, 242, and 250 based on a predetermined branch point 211. For example, the electronic device may determine, as the third reference point 241, a point at which a distance to the branch point 211 is minimum among intersection points formed between the contour 200 and vectors, the vectors being formed between a vector in a direction from the branch point 211 toward the first branch 203 and a vector in a direction from the branch point 211 toward the target branch 230. In addition, the electronic device may determine, as the second reference point 242, a point at which a distance to the branch point 211 is minimum among intersection points formed between the contour 200 and vectors, the vectors being formed between a vector in a direction from the branch point 211 toward the first branch 203 and a vector in a direction from the branch point 211 toward the other branch 220. Similarly, the electronic device may determine, as the first reference point 250, a point at which a distance to the branch point 211 is minimum among intersection points formed between the contour 200 and vectors, the vectors being formed between a vector in a direction from the branch point 211 toward the target branch 230 and a vector in a direction from the branch point 211 toward the other branch 220. In other words, the electronic device may determine, as the first reference point 250, a point at which a distance from a predetermined branch point 211 included in the branch region 210 where the plurality of second branches 220 and 230 branch from the first branch 203 to the contour of the target branch 230 is minimum. However, a method by which the electronic device determines the first reference point 250, the second reference point 242, the third reference point 241 is not limited thereto. The electronic device may set the other point 204 located farther from the target branch 230 than the second reference point 242 such that the second reference point 242 and the third reference point 241 are included. In other words, the electronic device may set the other point 204 as a boundary of the branch region 210 while including the second reference point 242 and the third reference point 241.

The electronic device according to an embodiment may select at least one of the plurality of second branches 220 and 230 to generate a virtual contour in the branch region 210 from the first branch 203 to the corresponding second branch.

When the electronic device according to an embodiment selects the target branch 230 among the plurality of second branches 220 and 230, the electronic device may generate a first candidate contour 252 by extending the contour of the target branch 230 from the first reference point 250 to the second reference point 242 that is a boundary between the other branch 220 and the first branch 203. For example, the electronic device may generate the first candidate contour 252 by linearly extending the contour of the target branch 230 from the first reference point 250 to the second reference point 242. Similarly, the electronic device may generate a third candidate contour 251 by extending the contour of the other branch 220 from the first reference point 250 to the third reference point 241 that is a boundary between the target branch 230 and the first branch 203. For example, the electronic device may generate the third candidate contour 251 by linearly extending the contour of the other branch 220 from the first reference point 250 to the third reference point 241.

However, when the electronic device extracts a vascular diameter of a path along the first branch 203 and the target branch 230 based on the first candidate contour 252, an excess region 270 in which the vascular diameter is overestimated depending on a structure of the target blood vessel may be included. Accordingly, the electronic device may generate one or more second candidate contours to reduce an error in the vascular diameter of the path along the first branch 203 and the target branch 230 caused by the excess region 270. A method by which the electronic device generates the second candidate contours will be described in detail below with reference to FIGS. 3 to 5.

FIG. 3 illustrates a second candidate contour generated by an electronic device based on a contour of a target blood vessel according to an embodiment.

An electronic device according to an embodiment (e.g., the electronic device 100 of FIG. 1) may extract a contour 300 of a target blood vessel. The electronic device may generate, among the extracted contour 300, a second candidate contour 330 by extending a predetermined first partial contour 320 of the target blood vessel in a direction from a first reference point 301 toward a second partial contour 310 of a first branch. For example, the electronic device may generate the second candidate contour 330 based on the first partial contour 320 and the second partial contour 310. For example, the electronic device may generate one or more second candidate contours 330 by extending the contour of the target branch (e.g., the first partial contour 320) to another point 305 located farther from the target branch than a second reference point (e.g., a point corresponding to a boundary between the other branch and the first branch, wherein the point is located at a minimum distance from the branch point to a lower contour). For example, the electronic device may generate the second candidate contour 330 in which the first partial contour 320 is extended to the second partial contour 310 by setting the length of the first partial contour 320 to 5 pixels and the length of the second partial contour 310 to 10 pixels and generating a Bezier curve. The electronic device may generate the second candidate contour 330 corresponding to a Bezier curve based on Equation 1 below. For reference, the Bezier curve represents a curve defined based on control points. For example, the Bezier curve is used to generate a curve connecting two different points. Accordingly, the electronic device may generate, based on Equation 1 below, the second candidate contour 330 connecting the first reference point 301 corresponding to an end point of the first partial contour 320 to the other point 305 corresponding to an end point of the second partial contour 310.

$$B ( t ) = {\sum }_{i = 0}^{k} ( \begin{matrix}k \\ i\end{matrix} ) {( 1 - t )}^{k - i} {t}^{i} {P}_{i} \qquad [ Equation 1 ]$$

In Equation 1, B(t) represents a point on a Bezier curve. Pᵢ represents control points. t represents a parameter greater than or equal to 0 and less than or equal to 1. k represents the value obtained by subtracting 1 from the total number of control points.

$$( \begin{matrix}k \\ i\end{matrix} ) {( 1 - t )}^{k - i}$$

represents the Bernstein coefficient. The electronic device may generate a curve by setting the first reference point 301 as a starting point and extending the first partial contour 320 toward the other point 305. For example, the electronic device may set P₀ as the first reference point 301 and Pₖ as the other point 305. The electronic device may extract an internal division point of a line segment connecting P₀ and Pₖ at a ratio of t:(1−t) as a new control point. The electronic device may extract a plurality of control points by recursively extracting internal division points between each Pᵢ. The electronic device may generate a Bezier curve by connecting these control points. Accordingly, the electronic device may generate the second candidate contour 330 based on Equation 1. The second candidate contour 330 generated by the electronic device may be expressed in a polynomial form based on Equation 2 below. In Equation 2 below, it is assumed that the second candidate contour 330 is a cubic Bezier curve.

$${b}_{0 , 3} = 1 - 3 t + 3 {t}^{2} - 1 {t}^{3} \qquad [ Equation 2 ]$$

$${b}_{1 , 3} = 0 + 3 t - 6 {t}^{2} + 3 {t}^{3}$$

$${b}_{2 , 3} = 0 - 0 t + 3 {t}^{2} - 3 {t}^{3}$$

$${b}_{3 , 3} = 0 + 0 t - 0 {t}^{2} + 1 {t}^{3}$$

Equation 2 may be expressed as a matrix multiplication equation as in Equation 3 below.

$${B}_{{P}_{0} , {P}_{1} , {P}_{2} , {P}_{3}} ( t ) = [ 1 , t , {t}^{2} , {t}^{3} ] [ \begin{matrix}1 & 0 & 0 & 0 \\ - 3 & 3 & 0 & 0 \\ 3 & - 6 & 3 & 0 \\ - 1 & 3 & - 3 & 1\end{matrix} ] [ \begin{matrix}- & {P}_{0} & - \\ - & {P}_{1} & - \\ - & {P}_{2} & - \\ - & {P}_{3} & -\end{matrix} ] \qquad [ Equation 3 ]$$

In Equation 3, B_{P}(t) represents a point on the Bezier curve calculated corresponding to a specific t value. [1,t,t²,t³] represents a vector constituting the Bezier polynomial. -Pᵢ- represents Bezier control points in a row vector format. The 4×4 matrix represents a transformation matrix including coefficients of the Bezier curve. Equation 3 above may be expressed as Equation 4 below.

$$V = TMP \qquad [ Equation 4 ]$$

In Equation 4, V∈ [symbol] ^{(j×d)} represents j points on the Bezier curve expressed in a row vector form. T= [symbol] ^{(j×k)} represents row vectors of t values raised to powers for j samples on the Bezier curve. M∈ [symbol] ^{(k×k)} represents a Bezier matrix. P∈ [symbol] ^{(k×d)} represents the control points in a row vector form. T in Equation 4 is defined as Equation 5 below, and M may be expressed by Equation 6 below.

$$T = [ \begin{matrix}{t}_{1}^{0} & {t}_{1}^{1} & … & {t}_{1}^{k} \\ {t}_{2}^{0} & {t}_{2}^{1} & … & {t}_{2}^{k} \\ & … & & \\ {t}_{j}^{0} & {t}_{j}^{1} & … & {t}_{j}^{k}\end{matrix} ] \qquad [ Equation 5 ]$$

In Equation 5, the T matrix may represent a matrix constituting polynomial terms of the Bezier curve. The T matrix may include j sampled points. The electronic device may calculate the Bezier curve at various t values based on the T matrix.

$$( \begin{matrix}k \\ i\end{matrix} ) {( 1 - t )}^{k - i} {t}^{i} = {\sum }_{p = 0}^{k} ( \begin{matrix}k \\ i\end{matrix} ) {( - 1 )}^{i - p} ( \begin{matrix}k - i \\ p - i\end{matrix} ) {t}^{p} \qquad [ Equation 6 ]$$

Equation 6 represents a lower triangular matrix corresponding to the Bezier matrix (M) of Equation 4. Equation 6 may express the lower triangular matrix as corresponding Bernstein polynomials.

Assuming that the electronic device according to an embodiment generates the second candidate contour 330 by fitting N points between the first reference point 301 and the other point 305, the electronic device may generate the second candidate contour 330 based on Equation 7 below. For reference, N represents a natural number equal to or greater than 1.

$$G = TMP \qquad [ Equation 7 ]$$

In Equation 7, G∈ [symbol] ^{(N×d)} represents a row-vector matrix corresponding to N points and d dimensions. For the electronic device to generate a (k−1)-th order Bezier curve composed of k control points, N>k+1 must be satisfied. Since the electronic device may have determined values of G, T, and M in advance, a Bezier control point P may be calculated based on Equation 8 below.

$${( TM )}^{- 1} G = P \qquad [ Equation 8 ]$$

Since a first control point P₀ and a last control point Pₖ of the Bezier curve must exist at both ends of the Bezier curve at t=0 and t=1, the electronic device may use a least square method to find the remaining control points by fixing P₀ and Pₖ. Assuming that N points are sampled at equal intervals, the T matrix may be expressed by Equation 9 below.

$${T}_{( j , i )} = {( \frac{n}{N} )}^{i} , n ∈ [ 0 … N ) , i ∈ [ 0 … k ) \qquad [ Equation 9 ]$$

In addition, since the Bezier matrix (M) may be defined based on Equations 4 and 6, Equation 10 below may be derived based on Equation 9 and the Bezier matrix (M).

$$C = TM ( C ∈ {ℝ}^{( N \times k )} ) \qquad [ Equation 10 ]$$

Furthermore, the electronic device may fix P₀ and Pₖ₋₁ at the first reference point 301 and the other point 305, respectively, and obtain the remaining control points P₁ to Pₖ₋₂ based on Equation 11 below.

$$G = CP \qquad [ Equation 11 ]$$

$$G = [ \begin{matrix}\vert & \vert & \vert \\ {C}_{0} & {C}_{1 … k - 2} & {C}_{k} - 1 \\ \vert & \vert & \vert\end{matrix} ] [ \begin{matrix}- & 0 & - \\ - & {P}_{1 … k - 2} & - \\ - & {P}_{k - 1} & -\end{matrix} ]$$

$$G = [ \begin{matrix}\vert & \vert \\ {C}_{0} & {C}_{k} - 1 \\ \vert & \vert\end{matrix} ] [ \begin{matrix}- & 0 & - \\ - & {P}_{k - 1} & -\end{matrix} ] + [ \begin{matrix}\vert \\ {C}_{1 … k - 2} \\ \vert\end{matrix} ] [ \begin{matrix}- & {P}_{1 … k - 2} & -\end{matrix} ]$$

$$G - [ \begin{matrix}\vert & \vert \\ {C}_{0} & {C}_{k} - 1 \\ \vert & \vert\end{matrix} ] [ \begin{matrix}- & {P}_{0} & - \\ - & {P}_{k - 1} & -\end{matrix} ] = [ \begin{matrix}\vert \\ {C}_{1 … k - 2} \\ \vert\end{matrix} ] [ \begin{matrix}- & {P}_{1 … k - 2} & -\end{matrix} ]$$

$${[ \begin{matrix}\vert \\ {C}_{1 … k - 2} \\ \vert\end{matrix} ]}^{+} ( G - [ \begin{matrix}\vert & \vert \\ {C}_{0} & {C}_{k} - 1 \\ \vert & \vert\end{matrix} ] [ \begin{matrix}- & 0 & - \\ - & {P}_{( k - 1 )} & -\end{matrix} ] ) = [ \begin{matrix}- & {P}_{1 … k - 2} & -\end{matrix} ]$$

In summary, the electronic device according to an embodiment may generate the second candidate contour 330 in which the first partial contour 320 is extended to the other point 305 by connecting the first reference point 301 to the other point 305 with a Bezier curve based on Equations 1 to 11 above.

FIG. 4 illustrates a second candidate contour generated by an electronic device in a lesion region according to an embodiment.

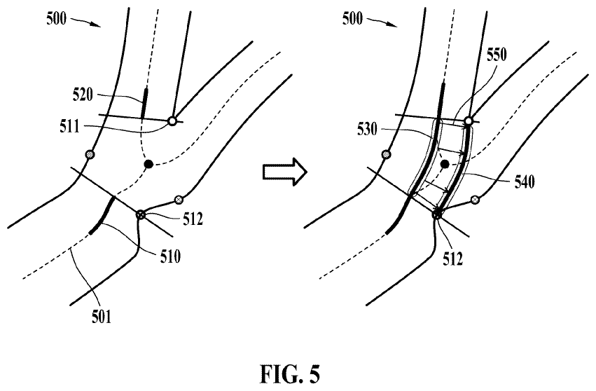

An electronic device according to an embodiment (e.g., the electronic device 100 of FIG. 1) may generate a virtual contour in a branch region based on a contour 400 of a target blood vessel. For example, the electronic device may generate a second candidate contour 440 in which a first partial contour 420 is extended to a second partial contour 410 such that the first partial contour 420 of the target blood vessel is connected to the second partial contour 410 of the first branch. In FIG. 4, a method by which the electronic device generates the second candidate contour 440 based on the contour 400 of the target blood vessel is the same as that illustrated in FIG. 3, and thus a redundant description thereof will be omitted. However, FIG. 4 illustrates a case in which the contour 400 of the target blood vessel includes a lesion region 430. When the lesion region 430 is present, the contour 400 of the target blood vessel may be locally deformed. In other words, the contour 400 in the lesion region 430 may be abnormally narrowed or irregular. Referring to FIG. 4, the second candidate contour 440 may be distorted by the lesion region 430. In other words, the electronic device may generate the second candidate contour 440 considering the shape of the lesion region 430. Accordingly, when the contour 400 includes the lesion region 430, a method of generating a second candidate contour that is less affected by the lesion region 430 than the method of generating the second candidate contour of FIG. 3 will be disclosed below with reference to FIG. 5.

FIG. 5 illustrates a second candidate contour generated by an electronic device based on a vascular centerline according to an embodiment.

An electronic device according to an embodiment (e.g., the electronic device 100 of FIG. 1) may generate a vascular centerline 501 of a target blood vessel based on a contour 500 of the target blood vessel. For example, the electronic device may calculate a midpoint between contours 500 facing each other among the contours 500. For example, the electronic device may generate a plurality of perpendicular lines in a direction perpendicular to a direction in which blood flows through the target blood vessel. The electronic device may calculate midpoints between intersection points of the perpendicular lines and the contour 500. The electronic device may generate the vascular centerline 501 by connecting the plurality of midpoints. The electronic device may generate one or more intermediate contours 530 by extending the vascular centerline of the target blood vessel (hereinafter, a first partial centerline 520) in a direction from a first reference point 511 toward another point 512. The electronic device may set the first partial centerline 520 to a length of 5 pixels and set a vascular centerline of the target blood vessel (hereinafter, a second partial centerline 510) to a length of 10 pixels. The electronic device may generate an intermediate contour 530 based on the first partial centerline 520 and the second partial centerline 510 through the Bezier curve generation method described with reference to FIG. 3. The electronic device may generate one or more second candidate contours 540 by transforming the one or more intermediate contours 530 in a direction 550 toward the contour 500 of the target blood vessel. For example, the electronic device may perform an affine transformation on the intermediate contour 530. For example, the electronic device may perform an affine transformation including translation, scaling, and rotation to move the intermediate contour 530 to a corresponding point of the contour 500 of the target blood vessel based on a start point (e.g., the first reference point 511) and an end point (e.g., the other point 512) of the intermediate contour 530.

FIG. 6 illustrates a plurality of candidate contours generated by an electronic device according to an embodiment.

An electronic device according to an embodiment (e.g., the electronic device 100 of FIG. 1) may extract a target blood vessel from a medical image 600. The electronic device may divide the target blood vessel for each path. For example, the electronic device may divide a target branch 605 from a first branch 603 as a first path. The electronic device may divide another branch 606 as a second path distinct from the first path. The electronic device may determine a first reference point 602 and a second reference point 601 in a contour of the target blood vessel. A method by which the electronic device determines the first reference point 602 and the second reference point 601 has been described above with reference to FIG. 2, and thus a redundant description thereof will be omitted. The electronic device may generate a first candidate contour 610 by linearly extending the contour of the target branch 605 from the first reference point 602 to the second reference point 601. In addition, the electronic device may generate a second candidate contour 620 by extending the contour of the target branch 605 from the first reference point 602 to another point 604 farther from the target branch 605 than the first reference point 602 using a Bezier fitting method. Furthermore, the electronic device may generate an intermediate contour 608 in a branch region based on a vascular centerline of the target blood vessel. The electronic device may generate another second candidate contour 630 by transforming (e.g., affine transformation) the intermediate contour 608 in a direction in which a virtual contour is to be generated. The electronic device may determine one of the generated candidate contours (e.g., the first candidate contour 610, the second candidate contour 620, and the other second candidate contour 630) as a target contour. Hereinafter, a method by which the electronic device determines the target contour will be described in detail.

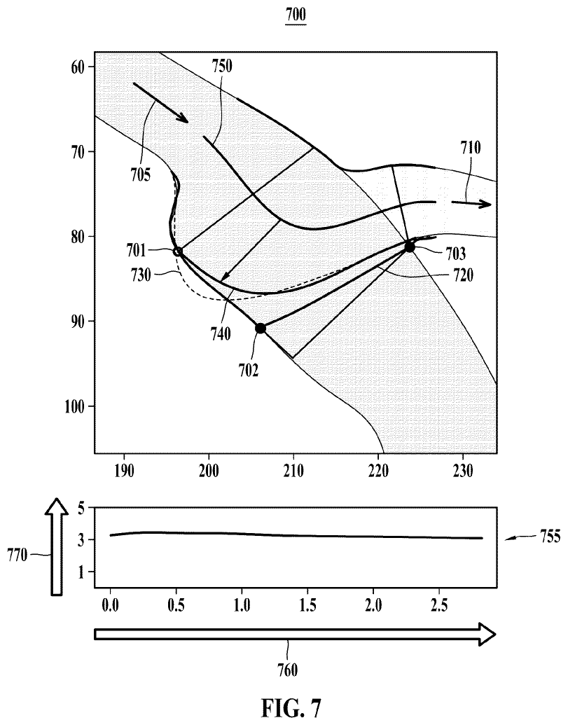

FIGS. 7 to 10 are diagrams for describing a method by which an electronic device determines a target contour according to an embodiment.

An electronic device according to an embodiment (e.g., the electronic device 100 of FIG. 1) may extract a target blood vessel from a medical image 700. The electronic device may divide the target blood vessel for each branch. For example, the electronic device may distinguish the plurality of branches based on their pixel values. Referring to FIG. 7, the electronic device may adjust a pixel value of a target branch 710 to be distinguished from a pixel value of the other branch. The electronic device may generate a plurality of candidate contours 720, 730, and 740 in a direction from a first branch 705 toward the target branch 710 in a branch region divided based on a predetermined other point 701. For example, the electronic device may generate a first candidate contour 720 connecting a first reference point 703 to a second reference point 702. For example, the electronic device may generate a second candidate contour 730 by extending the contour of the target branch 710 from the first reference point 703 to the other point 701. For example, the electronic device may generate an intermediate contour 750 based on a vascular centerline, and may generate another second candidate contour 740 based on transforming (e.g., affine transformation) the intermediate contour 750. The electronic device may determine a target contour corresponding to a path along the first branch 705 and the target branch 710 based on the first candidate contour 720 and the one or more second candidate contours (e.g., the second candidate contour 730 and the other second candidate contour 740). For example, the electronic device may determine whether the plurality of candidate contours 720, 730, and 740 is located inside the target blood vessel. For example, when at least one point constituting the second candidate contour 730 is located outside the target blood vessel, the electronic device may determine one of the remaining candidate contours (e.g., the first candidate contour 720 and the other second candidate contour 740) other than the second candidate contour 730 as the target contour. In other words, the electronic device may determine the target contour based on checking whether at least one point constituting each of the plurality of candidate contours 720, 730, and 740 is located inside the target blood vessel. Thereafter, the electronic device may determine, among the second candidate contours (e.g., the second candidate contour 730 and the other second candidate contour 740), a contour located inside the first candidate contour 720 as the target contour. Here, when there is a plurality of second candidate contours (e.g., the second candidate contour 730 and the other second candidate contour 740) located inside the first candidate contour 720, the electronic device may extract diameter information of the target blood vessel corresponding to the one or more second candidate contours (e.g., the second candidate contour 730 and the other second candidate contour 740) by scanning the target blood vessel in a direction from the first branch 705 toward the target branch 710. For example, when scanning the target blood vessel in the direction from the first branch 705 toward the target branch 710, the electronic device may draw a straight line perpendicular to a scanning direction. The electronic device may extract the diameter information of the target blood vessel based on a distance between a first intersection point formed between the perpendicular straight line and the contour of the target blood vessel in the branch region and a second intersection point formed between the perpendicular straight line and one of the plurality of candidate contours 720, 730, and 740. The electronic device may generate a graph 755 including a position in a direction from the first branch 705 toward the target branch 710 as a first axis 760 and the diameter information of the target blood vessel as a second axis 770. The electronic device may determine the target contour based on linearity of the graph 755 that changes along the first axis 760.

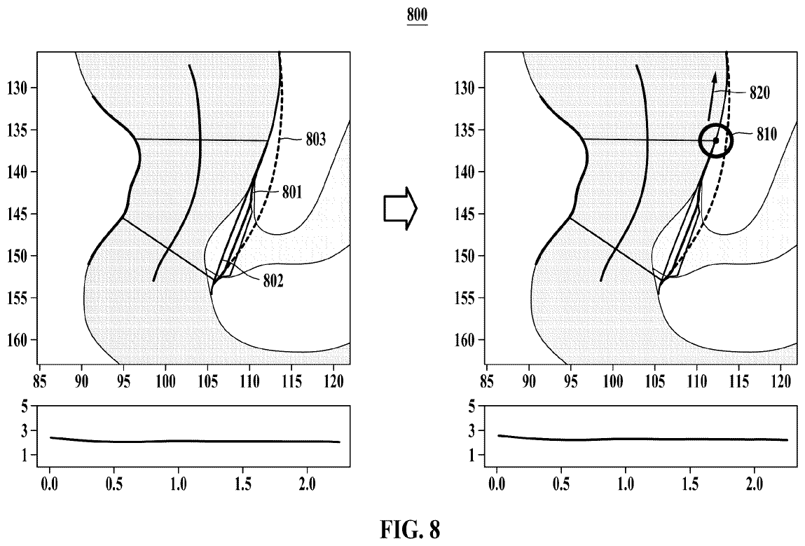

Referring to FIG. 8, an electronic device according to an embodiment (e.g., the electronic device 100 of FIG. 1) may generate a first candidate contour 801 and a plurality of second candidate contours 802 and 803 in a branch region of a target blood vessel in a medical image 800. For reference, the electronic device may generate one or more second candidate contours 802 of different shapes by moving another point 810 to which the second candidate contour 802 is connected. For example, in response to moving the other point 810 by a predetermined interval in a direction 820 of the first branch, the electronic device may generate one or more second candidate contours 802 by extending the contour of the target branch from the first reference point toward the moved other point. For example, the electronic device may move the other point 810 by an interval of 1 pixel in the direction 820 of the first branch and generate a corresponding Bezier curve by newly defining the moved other point 810 as an end point. The electronic device may consider the generated Bezier curve as the second candidate contour 802. The electronic device may generate one or more second candidate contours 802 while moving the position of the other point 810 by an interval of 1 pixel in the direction 820 of the first branch. The electronic device may determine the target contour based on the first candidate contour 801 and the plurality of second candidate contours 802 and 803. For example, the electronic device may exclude, from candidates for the target contour, a contour located outside the target blood vessel among the first candidate contour 801 and the plurality of second candidate contours 802 and 803. For example, the electronic device may exclude, from the candidates for the target contour, the second candidate contour 803 based on the vascular centerline, which is generated outside the target blood vessel. Thereafter, the electronic device may determine the target contour based on a result of comparing the first candidate contour 801 to the second candidate contour 802 generated based on the contour of the target blood vessel. For example, the electronic device may determine, as the target contour, a contour located more inside the target blood vessel between the first candidate contour 801 and the second candidate contour 802. Referring to FIG. 8, the electronic device may determine the second candidate contour 802 as the target contour.

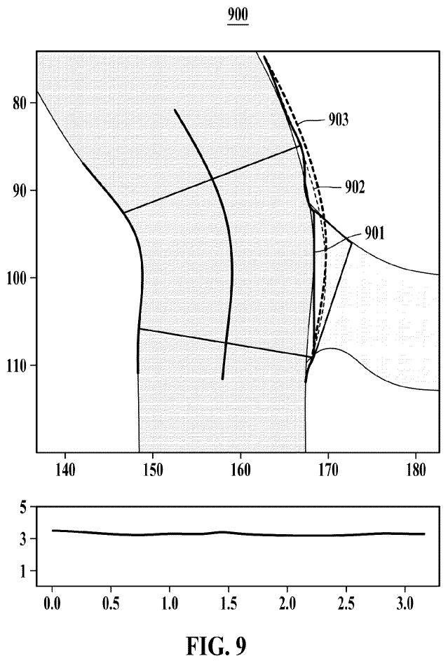

Referring to FIG. 9, an electronic device (e.g., the electronic device 100 of FIG. 1) may extract a T-shape target blood vessel in a medical image 900. The electronic device may generate a plurality of candidate contours 901, 902, and 903 in a branch region of the T-shape target blood vessel. For example, the electronic device may generate a first candidate contour 901 and a plurality of second candidate contours 902 and 903 in the branch region of the T-shape target blood vessel. In the T-shape target blood vessel, the electronic device may determine the target contour based on a positional relationship between the first candidate contour 901 and the plurality of second candidate contours 902 and 903. For example, when the electronic device determines that the plurality of second candidate contours 902 and 903 is located outside the first candidate contour 901, the electronic device may determine the first candidate contour 901 as the target contour. In other words, in the case of the T-shape target blood vessel, the electronic device may determine the first candidate contour 901 as the target contour with a high probability.

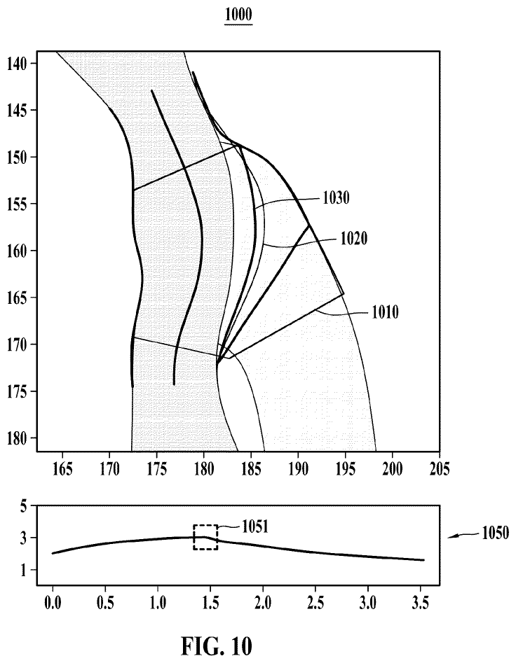

Referring to FIG. 10, an electronic device according to an embodiment (e.g., the electronic device 100 of FIG. 1) may determine, as the target contour, a candidate contour in which an extracted vascular diameter changes most linearly among the plurality of candidate contours. For example, the electronic device may generate a plurality of candidate contours 1010, 1020, and 1030 in a branch region of a target blood vessel included in a medical image 1000. The electronic device may determine whether the plurality of candidate contours 1010, 1020, and 1030 are located inside the target blood vessel. In the case of FIG. 10, the plurality of candidate contours 1010, 1020, and 1030 is located inside the target blood vessel. The electronic device may determine the target contour based on a positional relationship between the first candidate contour 1010 and the second candidate contours 1020 and 1030. For example, the electronic device may select the second candidate contours 1020 and 1030 located inside the first candidate contour 1010 as candidates for the target contour. When the plurality of second candidate contours 1020 and 1030 is located inside the first candidate contour 1010, the electronic device may extract diameter information of the target blood vessel corresponding to each of the second candidate contours 1020 and 1030 by scanning the target blood vessel in a direction from the first branch toward the target branch (e.g., in a downward direction from an upper portion of the target blood vessel in the blood vessel image of FIG. 10). The electronic device may generate a graph 1050 including a position in a direction of the target branch relative to the first branch as a first axis and the diameter information of the target blood vessel as a second axis. The electronic device may determine the target contour among the second candidate contours 1020 and 1030 based on linearity of the graph 1050. For example, the electronic device may compare linearity of the graph 1050 corresponding to each of the second candidate contours 1020 and 1030 and determine a contour corresponding to high linearity as the target contour. For example, the electronic device may exclude, from the candidates for the target contour, a candidate contour corresponding to the graph 1050 including a peak 1051 as illustrated in the graph 1050 of FIG. 10. However, a method by which the electronic device determines linearity of the second candidate contours 1020 and 1030 based on the graph 1050 is not limited thereto.

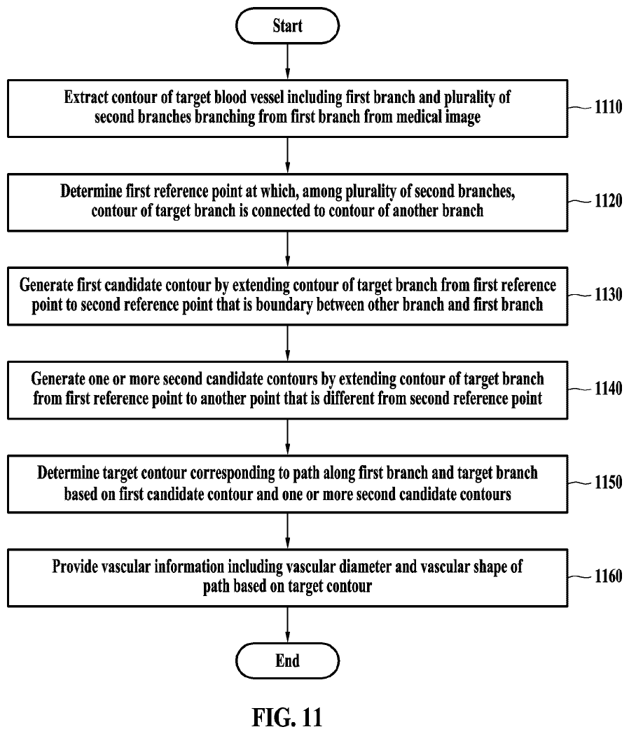

FIG. 11 is a flowchart of a method of processing a medical image, performed by an electronic device, according to an embodiment.

In operation 1110, the electronic device according to an embodiment may extract a contour of a target blood vessel including a first branch and a plurality of second branches branching from the first branch from a medical image. For example, the electronic device may identify a blood vessel in the medical image and detect an outline of the blood vessel by applying a masking technique.

In operation 1120, the electronic device according to an embodiment may determine a first reference point at which, among the plurality of second branches, a contour of a target branch is connected to a contour of another branch. For example, the electronic device may analyze a connection structure of the blood vessel based on a point located on the contour of the target blood vessel at a minimum distance from a branch point.

In operation 1130, the electronic device according to an embodiment may generate a first candidate contour by extending the contour of the target branch from the first reference point to a second reference point that is a boundary between the other branch and the first branch. For example, the electronic device may generate the first candidate contour by connecting the first reference point to the second reference point with a straight line.

In operation 1140, the electronic device according to an embodiment may generate one or more second candidate contours by extending the contour of the target branch from the first reference point to another point that is different from the second reference point. For example, the electronic device may generate the second candidate contour based on a Bezier curve based on the contour of the target blood vessel or a curve obtained by affine transforming a Bezier curve based on a vascular centerline.

In operation 1150, the electronic device according to an embodiment may determine a target contour corresponding to a path along the first branch and the target branch based on the first candidate contour and the one or more second candidate contours. For example, the electronic device may determine an optimal contour by analyzing whether the candidate contours are located inside the blood vessel, a positional relationship between the candidate contours, and linearity of vascular diameters corresponding to each of the candidate contours.

In operation 1160, the electronic device according to an embodiment may provide vascular information including a vascular diameter and a vascular shape of the path based on the target contour. For example, the electronic device may calculate an FFR based on the vascular information to evaluate a blood flow state in the blood vessel, and may provide the data to a medical analysis system or a surgical robot device in order to be used for diagnosis and treatment.

The examples described herein may be implemented using a hardware component, a software component and/or a combination thereof. A processing device may be implemented using one or more general-purpose or special-purpose computers, such as, for example, a processor, a controller and an arithmetic logic unit (ALU), a digital signal processor (DSP), a microcomputer, a field-programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is singular; however, one of ordinary skill in the art will appreciate that a processing device may include multiple processing elements and/or multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher-level code that may be executed by the computer using an interpreter.

The above-described hardware devices may be configured to act as one or more software modules in order to perform the operations of the above-described examples, or vice versa.

As used herein, “A or B”, “at least one of A and B”, “at least one of A or B”, “A, B or C”, “at least one of A, B and C”, and “at least one of A, B, or C,” each of which may include any one of the items listed together in the corresponding one of the phrases, or all possible combinations thereof.

A number of example embodiments have been described above. Nevertheless, it should be understood that various modifications may be made to these example embodiments. For example, suitable results may be achieved if the described techniques are performed in a different order, and/or if components in a described system, structure, device, or circuit are combined in a different manner, and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. An electronic device for medical image processing, the electronic device comprising:
an image acquisition unit configured to acquire a medical image;
memory configured to store the medical image and a plurality of instructions; and
a processor configured to execute the plurality of instructions stored in the memory,
wherein, when executing the plurality of instructions, the processor causes the electronic device to:
extract a contour of a target blood vessel including a first branch and a plurality of second branches branching from the first branch from the medical image,
determine a first reference point at which, among the plurality of second branches, a contour of a target branch is connected to a contour of another branch,
generate a first candidate contour by extending the contour of the target branch from the first reference point to a second reference point that is a boundary between the other branch and the first branch,
generate one or more second candidate contours by extending the contour of the target branch from the first reference point to another point that is different from the second reference point,
determine a target contour corresponding to a path along the first branch and the target branch based on the first candidate contour and the one or more second candidate contours, and
provide vascular information including a vascular diameter and a vascular shape of the path based on the target contour.

2. The electronic device of claim 1, wherein, when executing the plurality of instructions, the processor causes the electronic device to:
generate the one or more second candidate contours by extending the contour of the target branch from the first reference point to the other point located farther from the target branch than the second reference point.

3. The electronic device of claim 1, wherein, when executing the plurality of instructions, the processor causes the electronic device to:
generate the first candidate contour by linearly extending the contour of the target branch from the first reference point to the second reference point.

4. The electronic device of claim 1, wherein, when executing the plurality of instructions, the processor causes the electronic device to:
generate, based on a predetermined first partial contour of the target branch and a predetermined second partial contour of the first branch, the one or more second candidate contours by extending the predetermined first partial contour from the first reference point to the other point.

5. The electronic device of claim 1, wherein, when executing the plurality of instructions, the processor causes the electronic device to:
generate a vascular centerline of the target blood vessel based on the contour of the target blood vessel,
generate one or more intermediate contours by extending the vascular centerline of the target branch in a direction from the first reference point toward the other point, and
generate the one or more second candidate contours by transforming the one or more intermediate contours in a direction toward the contour of the target blood vessel.

6. The electronic device of claim 1, wherein, when executing the plurality of instructions, the processor causes the electronic device to:
in response to moving the other point by a predetermined interval in a direction of the first branch, generate the one or more second candidate contours by extending the contour of the target branch from the first reference point toward the moved other point.

7. The electronic device of claim 1, wherein, when executing the plurality of instructions, the processor causes the electronic device to:
determine, among the one or more second candidate contours, the target contour that is located inside the first candidate contour.

8. The electronic device of claim 7, wherein, when executing the plurality of instructions, the processor causes the electronic device to:
extract diameter information of the target blood vessel corresponding to the one or more second candidate contours by scanning the target blood vessel in a direction from the first branch toward the target branch, and
determine the target contour based on linearity of a graph including a position in the direction of the target branch relative to the first branch as a first axis and the diameter information of the target blood vessel as a second axis.

9. The electronic device of claim 1, wherein, when executing the plurality of instructions, the processor causes the electronic device to:
extract the contour corresponding to the target blood vessel by masking the target blood vessel in the medical image.

10. The electronic device of claim 1, wherein, when executing the plurality of instructions, the processor causes the electronic device to:
determine, as the first reference point, a point at which a distance from a predetermined branch point included in a branch region where the plurality of second branches branch from the first branch to the contour of the target branch is minimum, and
determine, as the second reference point, an intersection point located at a minimum distance from the branch point among intersection points formed between the contour of the target blood vessel and vectors disposed between a vector in a direction from the branch point toward the other branch and a vector in a direction from the branch point toward the first branch.

11. The electronic device of claim 1, wherein, when executing the plurality of instructions, the processor causes the electronic device to:
generate a vascular centerline of the target blood vessel based on the contour of the target blood vessel, and
determine a point at which the vascular centerline branches as a branch point.

12. The electronic device of claim 1, wherein, when executing the plurality of instructions, the processor causes the electronic device to:
calculate a Fractional Flow Reserve (FFR) corresponding to a blood vessel on the path based on the vascular information.

13. A method of processing a medical image, the method comprising:
extracting a contour of a target blood vessel including a first branch and a plurality of second branches branching from the first branch from a medical image;
determining a first reference point at which, among the plurality of second branches, a contour of a target branch is connected to a contour of another branch;
generating a first candidate contour by extending the contour of the target branch from the first reference point to a second reference point that is a boundary between the other branch and the first branch;
generating one or more second candidate contours by extending the contour of the target branch from the first reference point to another point that is different from the second reference point;
determining a target contour corresponding to a path along the first branch and the target branch based on the first candidate contour and the one or more second candidate contours; and
providing vascular information including a vascular diameter and a vascular shape of the path based on the target contour.

14. The method of claim 13, wherein the generating of the one or more second candidate contours comprises:
generating the one or more second candidate contours by extending the contour of the target branch from the first reference point to the other point located farther from the target branch than the second reference point.

15. The method of claim 13, wherein the generating of the first candidate contour comprises:
generating the first candidate contour by linearly extending the contour of the target branch from the first reference point to the second reference point.

16. The method of claim 13, wherein the generating of the one or more second candidate contours comprises:
generating, based on a predetermined first partial contour of the target branch and a predetermined second partial contour of the first branch, the one or more second candidate contours by extending the predetermined first partial contour from the first reference point to the other point.

17. The method of claim 13, wherein the generating of the one or more second candidate contours comprises:
generating a vascular centerline of the target blood vessel based on the contour of the target blood vessel;
generating one or more intermediate contours by extending the vascular centerline of the target branch in a direction from the first reference point toward the other point; and
generating the one or more second candidate contours by transforming the one or more intermediate contours in a direction toward the contour of the target blood vessel.

18. The method of claim 13, wherein the generating of the one or more second candidate contours comprises:
in response to moving the other point by a predetermined interval in a direction of the first branch, generating the one or more second candidate contours by extending the contour of the target branch from the first reference point toward the moved other point.

19. The method of claim 13, wherein the determining of the target contour comprises:
determining, among the one or more second candidate contours, the target contour that is located inside the first candidate contour.

20. A non-transitory computer-readable storage medium storing one or more computer programs including instructions for performing the method of claim 13.
